(19) United States
(12) Patent Application Publication
(10) Pub. No.: US 2018/0016543 A1
(43) Pub. Date: Jan. 18, 2018

(54) METHOD OF PRODUCING YEAST MUTANTS AND THE USE THEREOF

(71) Applicants: Erbslöh Geisenheim AG, Geisenheim (DE); BRAIN AG, Zwingenberg (DE)

(72) Inventors: Jürgen FRÖHLICH, Mainz (DE); Jürgen ECK, Bensheim-Auerbach (DE); Guido MEURER, Seeheim-Jugenheim (DE); Christian NAUMER, Mannheim (DE); Jörg BÜSCHER, Pfungstadt (DE); Jörg MAMPEL, Bensheim (DE)

(73) Assignees: Erbslöh Geisenheim AG, Geisenheim (DE); BRAIN AG, Zwingenberg (DE)

(21) Appl. No.: 15/548,983

(22) PCT Filed: Feb. 4, 2016
(86) PCT No.: PCT/EP2016/052437

§ 371 Date: Aug. 4, 2017

(30) Foreign Application Priority Data
Feb. 10, 2015 (EP) 15154520.9

(51) Int. Cl. C12N 1/18; C12G 1/022; C12N 15/01; C12R 1/865

(52) U.S. Cl. CPC C12N 1/18; C12R 1/865; C12G 1/0203; C12N 15/01; C12G 2200/11

(57) ABSTRACT

A method of producing yeast mutants and the use thereof. In order to provide yeasts which, at a given sugar content, produce a low ethanol content and a relatively high glycerol content in ethanolic fermentation and which are simultaneously obtainable rapidly, at least one yeast strain is contacted in a first mutagenesis step with a first mutagen and in a second mutagenesis step with a second mutagen. The first and second mutagens are different from one another and are selected from the following groups: nucleotide-alkylating agent, nucleotide-deaminating agent, and UV radiation. The method further includes a first selection step executed between the first and second mutagenesis steps and a second selection step being executed after the second mutagenesis step, in which the mutants that originate from the prior mutagenesis step in each case are exposed to a selection factor selected from the following groups: (a) hypertonic medium and (b) alcohol dehydrogenase inhibitor.

The present invention concerns a method for producing yeast mutants during which at least one yeast strain is contacted in a first mutagenesis step with a first mutagen and contacted in a second mutagenesis step with a second mutagen, and yeasts produced by this method and a use of such yeasts.

In recent years temperatures in many wine cultivation regions during the wine grape ripening period have risen sharply, which has also increased the sugar content of grapes ripened in this manner. In the case of ethanolic fermentation used to produce wine, this sugar, in particular glucose, is converted to ethanol as the predominant fermentation product through the use of yeast strains. In recent years, in the case of ethanolic fermentation for the production of wine, the problem has arisen that as a result of the increased glucose content in the grapes, wines have been produced which exhibited an elevated alcohol content. Consumers do not desire such an elevated alcohol content, though, as even in different vintages consumers prefer alcohol contents in individual wines which are as consistent as possible. Also demand for wines with a lower alcohol content has recently increased. Furthermore, ethanol in wines represents a component which on the one hand is necessary and desirable but, in the case of an elevated content, can lead to the wines' flavour qualities suffering. High glycerol concentrations, e.g. more than 10 g/l, have a positive effect on a wine's flavour properties.

An attempt at providing these properties consists of using known yeasts which produce less ethanol for the same quantity of sugar in must. Some such yeasts are known; particularly for production of high-quality wines it is, however, desirable to have a selection of different yeasts available which, instead of the ethanol, produce other, secondary substances which can substantially influence a wine's flavour properties.

Attempts to produce such yeasts in particular comprise intentional genetic modification and conventional cultivation and selection processes based on conventional cultivation. An example of such methods to produce yeasts with the desired properties is disclosed in WO 2011/080411. A disadvantage of specifically genetically modified organisms is that consumers are highly sceptical of these organisms. Cultivation methods and methods based on conventional cultivation and pure selection processes may also be feasible, but extremely protracted methods and consequently very expensive. Alternatively wild yeasts are also isolated and the desired properties tested. Such isolation is described in EP 2 634 247 131. This process too is very expensive and its success uncertain.

In this context the object of the invention consists of preparing yeasts which produce a lower ethanol content and a higher glycerol content during ethanolic fermentation for a given sugar content than an aforementioned strain used and which can simultaneously be obtained quickly.

This object is achieved by a method of the aforementioned type, whereby the first and the second mutagen differ from each other and are selected from the following group: nucleotide-alkylating agent, nucleotide-deaminating agent and UV radiation, and a first selection step is performed between the first and the second mutagenesis step and a second selection step is performed after the second mutagenesis step, whereby the mutants resulting from the respective preceding mutagenesis step are exposed to a selection factor which is selected from the following groups: (a) hypertonic medium and (b) alcohol dehydrogenase inhibitor.

It has been seen that in such a method, during which two mutagenesis steps are performed, whereby a selection step is additionally performed after each mutagenesis step, yeasts are produced which on the one hand provide a lower ethanol content during alcoholic fermentation for a given sugar concentration than the initial strain used and on the other produce a higher glycerol concentration under the same conditions. Both have a positive effect on the taste of a wine produced using such a yeast.

The term “yeast” as used here preferably refers to yeasts of the genus Saccharomyces, particularly preferably to the species Saccharomyces cerevisiae or Saccharomyces bayanus. This also includes subspecies such as Saccharomyces cerevisiae subsp. bayanus.

Diploid yeast strains are preferably used as the aforementioned strain for the first mutagenesis step in the method according to the invention. In the case of the mutations performed the diploidy is not lost, so that the yeast mutant finally produced is also diploid. An advantage of diploid yeasts is that their properties remain comparatively stable for generations and are therefore particularly suitable as pure yeasts. Precisely this property leads, however, to a non-specific mutation, triggered for example by radiation or mutagenic agents, to seem not meaningful. It has now surprisingly been seen that simply by combining such means for a non-specific mutation, namely nucleotide-alkylating agents, nucleotide-deaminating agents or UV radiation together with a selection and repeated mutation and further selection causes yeast mutants with desired properties, namely a reduced ethanol and increased glycerol production, to be produced.

The term “nucleotide-alkylating agent” describes a substance which forms a covalent bond between alkyl groups and DNA bases. Such modified bases can lead to base mispairs and thus to point mutations. Numerous substances are known which cause such alkylation.

The term “nucleotide-deaminating agent” describes a substance which splits amino groups from DNA bases. Such splitting also causes base mispairs, i.e. point mutations. Numerous substances are known which cause such deamination.

The term “UV radiation” as used here refers to electromagnetic radiation in the 400 nm to 100 nm wavelength range and in particular to UV-C radiation in a wavelength range of 290 nm to 100 nm. A wavelength range of 280 nm to 240 nm, in particular 254 nm, is preferred. A preferred radiation intensity comprises between 1000 μJ/cm² and 3000 μJ/cm², particularly preferably 2000 μJ/cm². The effect of such radiation on DNA causes the formation of pyrimidine dimers, in particular thymine dimers. These dimers influence DNA's three-dimensional structure and block the DNA polymerase during replication.

The term “hypertonic medium” as used here relates to a medium that contains an osmotically active substance, including a salt, sugar or sugar alcohol, in a concentration which causes an osmolarity of more than 308 mOsmol/l. The medium is present in liquid form or in a solidified form through the addition of agar. Suitable osmotically active substances are known to the skilled person.

An “alcohol dehydrogenase inhibitor” is a substance which is capable of inhibiting the alcohol dehydrogenase enzyme, i.e. to block its activity so that no more acetaldehyde is converted by the enzyme which acts as the catalyst for converting acetaldehyde into ethanol in yeast. The inhibitor itself is not converted. Numerous substances which have this property are known to the skilled person.

In an embodiment an exhaustive test step is performed in which yeast mutants obtained in the method are tested and selected for whether they produce more glycerol and less ethanol during ethanolic fermentation than the aforementioned yeast strain used under the same conditions.

It shall be understood that the same conditions means that the yeast mutants and the aforementioned yeast strain used are incubated in the same medium, the same atmosphere and at the same temperature, as simultaneously as possible and the concentration of glycerol and ethanol is then determined. Suitable methods of determining the glycerol concentration are known to the skilled person. Media used for the test step are preferably selected from grape must obtained from wine grapes and an artificial must medium which imitates the conditions of a grape must. Various such artificial must media are known to the skilled person.

In an embodiment, the nucleotide-alkylating agent is selected from the following: dimethyl sulphate (DMS), ethyl methanesulphonate (EMS), methyl methanesulphonate (MMS), 1-methyl-3nitro-1-nitrosoguanidine (MNNG), methylnitrosocyanamide (MNC), methylnitrosourea (MNU) and DNA methyltransferases. It is known of all these substances that they alkylize DNA bases and it is recognised that these are especially suitable for mutagenesis in the case of Saccharomyces cerevisiae and Saccharomyces bayanus. Ethyl methanesulphonate (EMS) is preferably used for the method according to the invention.

In an embodiment, the nucleotide-deaminating agent is selected from the following: anorganic nitrite salt, organic nitrite salt and nitrous acid. It has been seen that these nucleotide-deaminating agents create a sufficiently mutagenic condition for the mutation of Saccharomyces cerevisiae and Saccharomyces bayanus, which on the other hand, however, facilitates their survival. A sodium nitrite is preferably used for the method according to the invention.

In an embodiment, the first mutagen is a nucleotide-alkylating agent or a nucleotide-deaminating agent. It has been seen that under these conditions a great number of yeast mutants can be obtained after the first mutagenesis step and selection step, which can be used for further mutagenesis.

In an embodiment, the first mutagen is a nucleotide-alkylating agent and the second mutagen is a nucleotide-deaminating agent or UV radiation. It was possible to obtain particularly large numbers of yeast mutants which can be used in a second mutagenesis step through the special combination of a nucleotide-alkylating agent as first mutagen. The second mutagen is preferably a nucleotide-deaminating agent.

In an embodiment, the hypertonic medium is obtained by addition of one of the following substances: chloride and sulphate of sodium, potassium, magnesium, calcium and sugar, including fructose and glucose, and sugar alcohols, including sorbitol and mannitol. It is known to the skilled person that in the context of yeast the term hypertonic relates to the fact that the hypertonic aqueous solution has a higher osmotic pressure than the yeast cytoplasm, i.e. it has an osmolarity of more than 308 mOsmol/l. An osmolarity in the range of 500 to 700 mOsmol/l is preferred.

In an embodiment, the alcohol dehydrogenase inhibitor is selected from the following: pyrazole, 3methylpyrazole, 4-methylpyrazole and acetylsalicylic acid. It has been seen that these alcohol dehydrogenase inhibitors are particularly suitable for the selection of yeast, including Saccharomyces cerevisiae and Saccharomyces bayanus, for which a reduced ethanol production is desired. The use of pyrazole is particularly preferred.

In an embodiment, one of the selection factors is selected from Group (a) and one of the selection factors is selected from Group (b). In the method according to the invention, more mutants can be produced by combining two different selection factors, which have better properties regarding a reduced ethanol production and increased glycerol production.

In an embodiment, the first selection factor is selected from Group (a) and the second selection factor is selected from Group (b).

In another embodiment, the first selection factor is selected from Group (b) and the second selection factor is selected from Group (a).

In an embodiment, a test step is performed after the first selection step, during which intermediate yeast mutants obtained after the first selection step are tested to see whether they produce more glycerol during an ethanolic fermentation under the same conditions as the aforementioned yeast strain used, whereby only such intermediate yeast mutants to which this applies are subjected to the second mutagenesis step and the second selection step.

It is preferably also tested whether the intermediate mutants which produce more glycerol also produce less ethanol during ethanolic fermentation than the aforementioned yeast strain used, whereby only such intermediate yeast mutants which produce more glycerol and less ethanol are subjected to the second mutagenesis step and the second selection step.

It shall be understood that the same conditions mean that the intermediate yeast mutants and the aforementioned yeast strain used are incubated in the same medium, the same atmosphere and at the same temperature as simultaneously as possible and the concentration of glycerol and preferably also ethanol is subsequently determined. Suitable methods for determining glycerol concentration and for determining the ethanol concentration are known to the skilled person.

The aforementioned objective is also achieved by a yeast mutant obtained according to a method described above and deposited with the Leibnitz-Institut DSMZ-Deutsche Sammlung von Mikroorganismen und Zellkulturen GmbH under accession number DSM 29822. This is a Saccharomyces cerevisiae subsp. bayanus, taxonomic designation: Saccharomyces cerevisiae. The reference sign used by the applicant is NP12B8 or herein also Nitrite Pyra 12 B8 or NO2Pyra12B8.

The strain is propagated in a medium with 10 g yeast extract, 10 g Bacto Peptone, 5 g NaCl made up to 1 l with H₂O and whose pH value is set at 7. For this the medium is sterilised for 20 minutes at 121° C. and after sterilising the pH value is between 6 and 7. The propagation takes place aerobically at a temperature of 30° C. Incubation takes place for 24 hours.

When must from Riesling grapes which has a must weight of 90° Oe (21.6% Brix) obtained by chaptalization, with a NOPA value of 107 mg/ml (+/−5 mg/l), for a yeast dosing of 4×10⁶/ml after 35 days at a fermentation temperature of 15-25° C., this yeast mutant produces a wine with the following proportions of ethanol, glucose, fructose, succinic acid and glycerol:
Ethanol: 90-106 g/l
Glucose: 0.18-0.42 g/l
Fructose: 0.95-5.55 g/l
Succinic acid: 2-5 g/l
Glycerol: 10-16 g/l

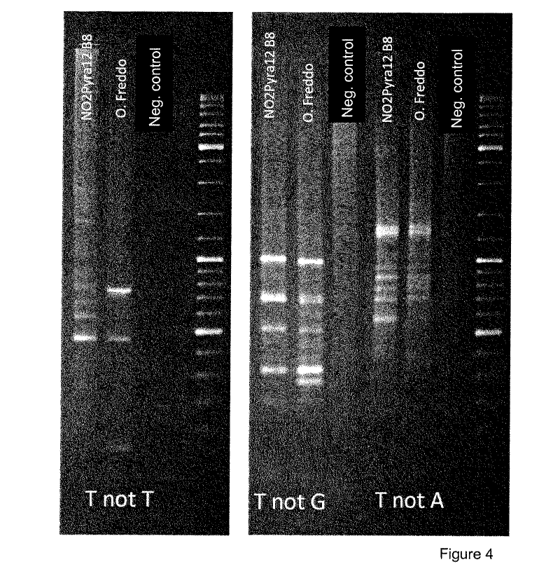

This yeast mutant is further characterised by the fact that is has the characteristic DNA profile shown in FIG. 4 described in a verification procedure in DE 10 2006 022 569, which is generally described there for microorganisms, using the primers A-not, C-not, G-not, T-not described there in a first PCR reaction and use of the primers T-not-A, T-not-T, T-not-G described there in a second PCR reaction and subsequent gel electrophoresis also described in DE 10 2006 022 569.

In the process “NO2Pyra1268” designates in FIG. 4 the yeast deposited with the Leibnitz-Institut DSMZ-Deutsche Sammlung von Mikroorganismen und Zellkulturen GmbH under accession number DSM 29822, “O. Freddo” designates the Saccharomyces cerevisiae subsp. bayanus strain LW 317-30, which is commercially available worldwide under the designation “Oenoferm Freddo F3” and “Neg. control” designates a sample which contained no DNA. The GeneRuler DNA Ladder Mix from Thermo scientific (Fermentas) was used as the length standard.

The problem described initially is also solved by a yeast mutant obtained according to a method described above, whereby when fermenting must at 90° Oe (21.6% Brix) and a NOPA value=107 mg/l (+/−5 mg/l) with a dosing of 4×10⁶/ml, after 35 days at a fermentation temperature of 15-25° C. the mutant produces a wine with the following proportions of ethanol and glycerol:

| | | |
|---|---|---|
| Ethanol | 70-150 | g/l |
| Glycerol | 10-20 | g/l |

The aforementioned problem is furthermore solved by the use of a yeast mutant obtained according to the method described above or the yeast mutant deposited with the Leibnitz-Institut DSMZ-Deutsche Sammlung von Mikroorganismen and Zellkulturen GmbH with accession number DSM 29822, or a yeast mutant obtained according to a method described above, whereby when fermenting must at 90° Oe (21.6% Brix) and a NOPA value=107 mg/l (+/−5 mg/l) with a dosing of 4×10⁶/ml, after 35 days at a fermentation temperature of 15-25° C. the yeast produces a wine with the following proportions of ethanol and glycerol:

| | | |
|---|---|---|
| Ethanol | 70-150 | g/l, |
| Glycerol | 10-20 | g/l, |

in a method for production of an alcoholic beverage.

In an embodiment, the use is such that the alcoholic beverage is produced from grape must. It shall be understood that it comprises both use for production of an alcoholic beverage from must from white and red grapes, including white must obtained from red grapes by the saignée method (blanc de noir) and rosé versions thereof.

Other advantages, features and potential applications of the present invention are clear from the following description of preferred embodiments and examples.

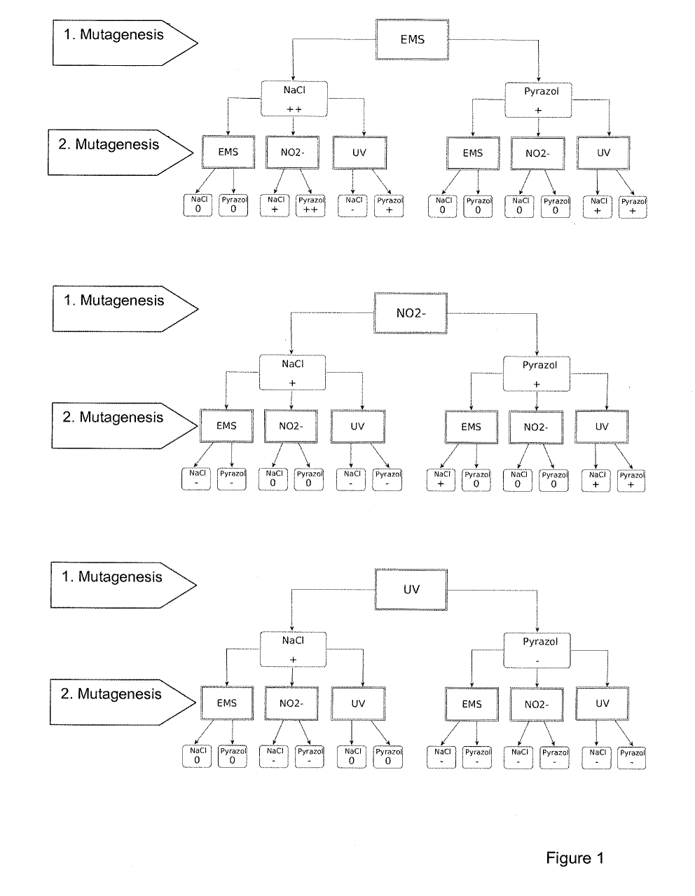

FIG. 1: Diagram of mutagenesis and selection steps

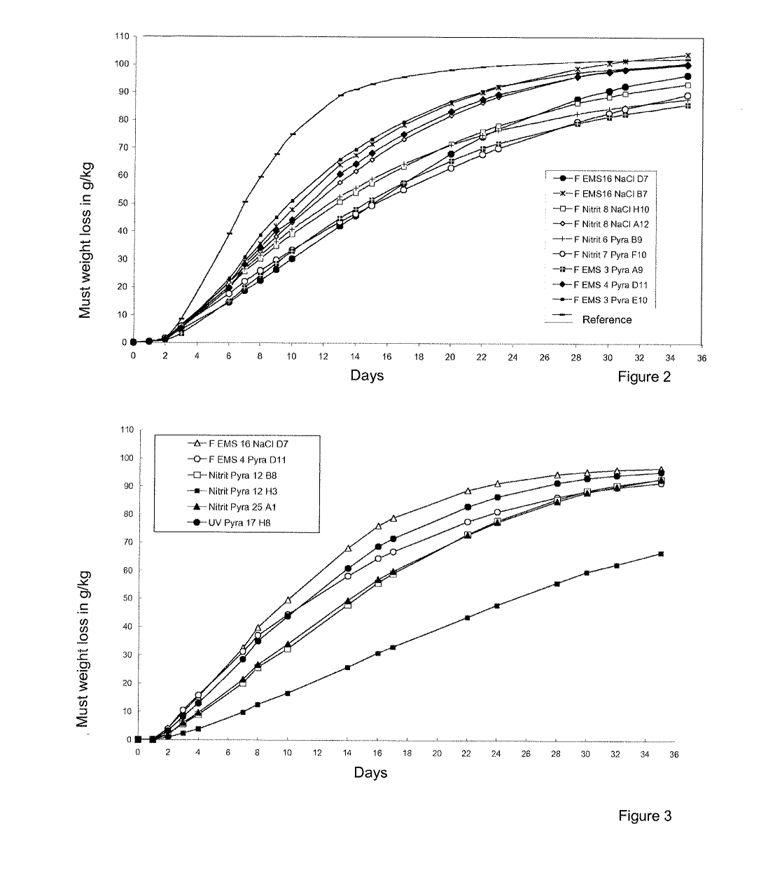

FIG. 2: Fermentation progress after a first mutagenesis and a first selection step

FIG. 3: Fermentation progress after a second mutagenesis and a second selection step

FIG. 4: DNA profile of the yeast mutant deposited under access number: DSM29822

### EXAMPLES

Various mutation tests were performed to produce yeast mutants, which produce a low ethanol concentration and increased glycerol concentration in a wine or a medium at a specified initial glucose concentration.

Preliminary Tests

In the initial tests performed using ethidium bromide, which produces mutations through frameshifts, it was possible to show that in the process only a few viable yeasts could be produced, which are in particular compromised with regard to their respiratory chain and appear to show general changes to the mitochondrial DNA. Such yeasts are unsuitable for a fermentation.

It was further possible to show that a repeated use of the same mutagen leads to only a few or no yeast mutants at all being obtained after the second mutagenesis step and subsequent selection.

The mutagenesis schemes shown in FIG. 1 were performed, whereby “++” means that more than 100 yeast mutants with the desired properties were obtained, “+” means that between 1 and 99 yeast mutants with the desired properties were obtained, “−” means that no yeast mutants with desired properties were obtained and “0” means that no yeast mutants at all were obtained. By desired properties it is understood here that the mutants produce more glycerol and less ethanol during an ethanolic fermentation than the aforementioned yeast strain used under the same conditions.

It was possible to determine during test fermentations that the production of glycerol generally takes place immediately after fermentation starts and customarily lasts up to 10 days. The majority of the glycerol is produced by fermentation of the first 100 g sugar per litre of medium, thereafter glycerol production slows, but in principle does not stop completely.

In contrast, it has been seen that the ethanolic fermentation which produces ethanol lasts up to three weeks, so that ethanolic fermentation and glycerol production slightly overlap during fermentation.

The different mutagenesis and/or selection steps and test methods are explained below in detail.

General Overview

The mutagenesis and selection steps were selected such that they are based on the production method for wine.

After the first mutagenesis and first selection 10,000 mutants were selected and tested for their glycerol production. Of the 10,000 mutants screened in this way, 400 were selected which have the highest glycerol concentration and tested again for their glycerol production.

Mutants which have a reproducible increased glycerol production were subjected to a small-scale wine fermentation and then organoleptically analysed. Ethanol determination was also carried out.

The mutants with the best organoleptic profile, the highest glycerol production, the lowest ethanol concentration and the best fermentation capacity were selected for the second mutagenesis step. In the process, the organoleptic profile was subjectively determined by tasting and evaluation of oxidative note, acidity, bitterness and overall impression. The fermentation capacity ensues from the fermentation speed, measured by a weight loss during fermentation and the fermentation's duration until the sugar present, in particular glucose, is consumed.

A mutant was then regarded as suitable when it had converted at least 70% of the sugar present after 35 days.

Mutagenesis Step With a Nucleotide-Alkylating Agent

Ethyl methanesulphonate (EMS) was used as the nucleotide-alkylating agent. A colony of a yeast strain was inoculated from agar plates into 5 ml YPD medium and cultured overnight at 28° C. The cells were then pelleted by centrifugation and washed twice with 100 mM potassium phosphate buffer with a pH value of 7. The cells were then resuspended in 10 ml of a 100 mM potassium phosphate buffer at pH value 7. 37.5 μl of pure EMS was added to 500 μl of the cell suspension. The suspension was incubated for an hour at 30° C. on a shaker. The reaction was stopped by the addition of 1 ml 5% sodium thiosulphate (% by weight). The yeast cells were then washed once with a 5% aqueous solution of sodium thiosulphate. After centrifugation the pellet was resuspended into 500 μl YPD medium.

Mutagenesis Step With a Nucleotide-Deaminating Agent

Sodium nitrite was used as a nucleotide-deaminating agent. Colonies of yeast strains were inoculated from agar plates into 5 ml YPD medium and cultured overnight at 28° C. The cells were pelleted by centrifugation and washed twice with 2 ml water. After washing, the cells were resuspended in a mixture of 2 ml water, 2 ml of a 0.6 M sodium acetate buffer with a pH of 4.5 and 2 ml of a 55 ml sodium nitrite solution. The cell suspension was incubated on a shaker for 8 minutes at 30° C. After incubation, 1 ml of the suspension was mixed with 9 ml of a 0.67 M potassium phosphate buffer with a pH value of 7 to stop the reaction.

Mutagenesis Step With UV Radiation

Colonies of yeast strains were inoculated from agar plates into 5 ml YPD medium, which contained 0.3 M sodium chloride and cultured overnight at 28° C. The cells were pelleted by centrifugation and washed once in 10 ml KP medium without glucose and fructose and resuspended. The optical density (OD) was measured at 600 nm and the suspension was diluted to an optical density of 0.025 and transferred to a Petri dish. The cell suspension in the petri dish was then irradiated with UV radiation of 254 nm wavelength at an intensity of 2000 μJ/cm² for 45 seconds in a Hoefer UVC 500 Crosslinker.

Selection Step With Alcohol Dehydrogenase Inhibitor

Pyrazole was used for selection with an alcohol dehydrogenase inhibitor.

For this selection step, the yeast cells obtained from a mutation step were spread on plates with KP medium which also contained 5 g/l pyrazole, whereby approximately 2,000 to 3,000 cells were transferred to a plate measuring 30×30 cm. The plates were incubated for 10 days at 18° C. under microaerophilic conditions. Microaerophilic conditions designates that the gas mixture (atmosphere) surrounding the plates had only 2 to 10% by volume of oxygen instead of the 20% by volume of oxygen which is otherwise normal for air.

Selection Step With Hypertonic Medium

In this selection step, sodium chloride was used to cause osmotic stress. The yeast cells obtained from a mutagenesis step were transferred to plates with KP medium, which also contained 17.53 g/l sodium chloride, whereby approximately 2,000 to 3,000 cells were applied to a plate measuring 30×30 cm. It is generally assumed that mutants which have a growth advantage on hypertonic media produce more glycerol. The plates were incubated for 10 days at 18° C. under microaerophilic conditions. Microaerophilic conditions designates that the gas mixture (atmosphere) surrounding the plates had only 2 to 10% by volume of oxygen instead of the 20% by volume of oxygen which is otherwise normal for air.

Media

The KP medium used, a medium which is also designated as artificial must, which was used, inter alia, for the selection steps, contained 115.5 g glucose monohydrate, 105 g fructose, 3 g tartaric acid, 0.3 g citric acid, 0.3 g malic acid, 0.3 g (NH₄)₂SO₄, 2 g KH₂PO₄, 0.2 g MgSO₄×7H₂O, 4 mg MnSO₄×H₂O, 4 mg ZnSO₄×7 H₂O, 0.5 mg CuSO₄×5 H₂O, 0.5 mg Kl, 0.2 mg CoCl₂×6 H₂O, 0.5 mg (NH₄)₆Mo₇O₂₄, 0.5 mg H₃BO₃, 300 mg myoinositol, 1 mg nicotinic acid, 1 mg calcium pantothenate, 1 mg pyridoxine hydrochloride, 0.04 mg biotin, 1 mg p-aminobenzoic acid, 247 mg L-glutamine, 183 mg L-arginine, 87.7 mg L-tryptophan, 71 mg L-alanine, 58.9 mg L-glutamic acid, 38.4 mg L-serine, 37.1 mg L-threonine, 23.7 mg L-leucine, 21.8 mg L-aspartic acid, 21.8 mg L-valine, 18.6 mg L-phenylalanine, 16 mg L-isoleucine, 16 mg L-histidine, 15.4 mg L-methionine, 9 mg L-tyrosine, 9 mg L-glycine, 8.3 mg L-lysine and 6.4 mg L-cysteine per litre.

The YPD medium contained 10 g yeast extract, 20 g peptone and 20 g glucose per litre and was set at a pH value of 5.5 to 6.0.

15 g/l agar was added to the media for plates with the media described.

Investigation of Glycerol Production

Yeast colonies from an agar plate were inoculated into YPD medium to determine glycerol production. After three days' growth at 30° C., 1 ml KP medium was inoculated with 20 μl of the culture grown in this way. There was no adjustment of the cell density. The KP cultures were then incubated at 18° C. under microaerophilic conditions. After ten days the excess culture was removed by centrifugation and filtration. The excess was then analysed with regard to the glycerol concentration. Determination of the glycerol concentration was carried out photometrically. A kit from R-Biopharm AG was used for the measurement, whereby the test was adjusted for measurement in a microtitration plate to a total sample volume of 155 μl.

Fermentation Tests

A real must was used for the fermentation tests, not an artificial must, whereby a 2013 vintage Riesling with 70° Oe (17.1% Brix) enriched by addition of saccharose (52 g) to 90° Oe (21.6% Brix) with a NOPA value of 107 mg/l (+/−5 mg/l) was used.

For the fermentation, whose results are shown in FIG. 2, a 2013 vintage Riesling must was used, which was enriched to 91° Oe (21.7% Brix) and which had a NOPA value of 124 mg/l (+/−5 mg/l).

The media pH value was between 3.1 and 3.2. In every case, the fermentation temperature was 18° C. The fermentation trials were not stirred.

Determination of Glycerol, Ethanol, Glucose and Fructose Produced and Total Sugar/Organoleptic Analysis/Fermentation Capacity

Analysis of the test fermentations was carried out by HPLC with the following parameters:

Equipment: Ultimate 3000 ThermoScientific

Column REZEX ROA Organic Acid H+ Phenomenex

RI Detector & UV detector at 210 nm

Solvent mixture: 0.005 N H₂SO₄ (isocratic)

Temperature: 75° C.

Flow rate: 0.5 ml/min

The organoleptic profile, which also results from the wine's composition, was also subjectively determined by tasting and evaluation of the oxidative note, acidity, bitterness and overall impression.

The fermentation capacity ensues from the fermentation speed, measured by a weight loss during fermentation and the fermentation duration, until the sugar present, in particular glucose, is consumed.

### Example 1

To determine the various effects of different mutagenic stimuli, tests were first carried out in which a Saccharomyces cerevisiae subsp. bayanus strain was exposed to UV radiation, EMS or sodium nitrite. In the case of the strain used in this example, it was the yeast strain available worldwide under the designation “Oenoferm Freddo F3”.

This was followed by selection either through a hypertonic sodium chloride medium or on a pyrazole medium.

FIG. 2 shows the results of investigation of some strains which resulted from the mutation with EMS or sodium nitrite. FIG. 2 shows in a graph the weight loss during a test fermentation over a total of 35 days, whereby the total weight of the must used was determined by weighing and the weight-loss data relates to the weight of the must with yeast originally used. The greater the weight loss, the better the respective strain's fermentative capacity.

Table 1 below shows the results of the HPLC analysis of this test fermentation after 35 days, whereby two independent fermentation trials were investigated for each mutant.

TABLE 1

| | Glucose | Fructose | Total sugar | Ethanol | Glycerol |
|---|---|---|---|---|---|
| Sample | g/l | g/l | g/l | g/l | g/l |
| F EMS16 NaCl D7 | 2.53 | 14.83 | 17.36 | 100.2 | 8.97 |
| F EMS16 NaCl D7 | 0.26 | 4.28 | 4.54 | 105.0 | 9.75 |
| F EMS16 NaCl B7 | 0.29 | 4.62 | 4.91 | 105.7 | 6.57 |
| F EMS16 NaCl B7 | 0.02 | 0.23 | 0.25 | 107.1 | 6.57 |
| F Nitrite 8 NaCl H10 | 0.07 | 1.38 | 1.45 | 107.1 | 6.64 |
| F Nitrite 8 NaCl H10 | 6.66 | 26.25 | 32.91 | 92.6 | 6.24 |
| F Nitrite 8 NaCl A12 | 0.04 | 1.31 | 1.35 | 106.4 | 6.75 |
| F Nitrite 8 NaCl A12 | 0.38 | 4.82 | 5.20 | 107.1 | 6.51 |
| F Nitrite 6 Pyra B9 | 12.04 | 39.09 | 51.13 | 85.2 | 5.84 |
| F Nitrite 6 Pyra B9 | 0.03 | 0.60 | 0.63 | 111.4 | 6.88 |
| F Nitrite 7 Pyra F10 | 3.59 | 19.85 | 23.44 | 100.2 | 6.54 |
| F Nitrite 7 Pyra F10 | 3.56 | 19.31 | 22.87 | 98.1 | 6.57 |
| F EMS 3 Pyra A9 | 18.37 | 45.85 | 64.22 | 79.8 | 5.72 |
| F EMS 3 Pyra A9 | 0.09 | 2.34 | 2.43 | 109.2 | 6.93 |
| F EMS 4 Pyra D11 | 0.26 | 4.03 | 4.29 | 105.7 | 8.06 |
| F EMS 4 Pyra D11 | 0.00 | 0.31 | 0.31 | 108.5 | 8.14 |
| F EMS 3 Pyra E10 | 0.00 | 0.77 | 0.77 | 109.2 | 7.09 |
| F EMS 3 Pyra E10 | 0.00 | 2.55 | 2.55 | 110.7 | 7.01 |
| Reference | 0.00 | 0.21 | 0.21 | 112.1 | 7.17 |
| Reference | 0.00 | 0.19 | 0.19 | 109.9 | 7.25 |

In FIG. 2 and Table 1 the yeasts which were first subjected to a mutagenesis step with EMS and then a selection with NaCl are designated “F EMS 16 NaCl E7” and “F EMS 16 NaCl B7”. Strains which were first exposed to a mutagenesis step with nitrite and a selection with NaCl are designated “F Nitrite 8 NaCl H10” and “F Nitrite 8 NaCl A12”, strains which were first subjected to a mutagenesis step with nitrite and then a selection with pyrazole are designated “F Nitrite 6 Pyra B9” and “F Nitrite 7 Pyra F10” and strains which were first subjected to a mutagenesis step with EMS and then a selection with pyrazole are designated “F EMS 3 Pyra A9”, “F EMS 4 Pyra D11” and “F EMS 3 Pyra E10”. The designation “Reference” refers to the Saccharomyces cerevisiae subsp. bayanus strain used as a comparison yeast, which was also used for the aforementioned first mutagenesis step.

It can be seen that as a result of this first mutagenesis step and subsequent selection step mutants are obtained which both produce less and more glycerol than the comparison yeast under the same conditions. Basically, it must be taken into consideration, though, that a part of the intermediate mutants had not converted all the sugar after 35 days, so that the glycerol values in these tests are comparable only to limited extent.

### Example 2

In this specimen test the “F EMS 16 NaCl D7” strain obtained from Example 1, which showed a particularly high glycerol concentration in the test fermentation, was selected for a second mutagenesis step and selection step. EMS, sodium nitrite and UV radiation were also used for the second mutagenesis step. A subsequent selection in each case was done with sodium chloride or pyrazole. It was seen that in the case of the strain previously mutated with EMS, no viable mutants were obtained in a further mutation with EMS.

The results of a fermentation test with different mutants obtained after the second mutagenesis step and the second selection are shown as an example in FIG. 3.

“F EMS 16 NaCl D7” and “F EMS 4 Pyra D11” designate intermediate mutants. In this example, they represent comparison values.

“Nitrite Pyra 12 B8”, “Nitrite Pyra 12 H3” and “Nitrite Pyra 25 Al” designate strains for which the second mutagenesis step was done with sodium nitrite and the second selection step with pyrazole. UV Pyra 17 A8 designates a strain for which the second mutagenesis step was done with UV radiation and the second selection with pyrazole.

Table 2 shows the results of the HPLC investigations of these strains. Here too it can be seen that some strains were obtained which produce much more glycerol than the intermediate mutants obtained in Example 1 and some strains were obtained which produce less glycerol. In total only a few mutants were produced which produce much less ethanol than the aforementioned intermediate mutants used at the start of the second mutagenesis step. The “Nitrite Pyra 12 H3” strain is characterised by neither glucose nor fructose being fully converted, which indicates an incomplete fermentation.

The data shown in Table 2 were obtained after 35 days of test fermentation, whereby two fermentation trials for each mutant were performed and evaluated independently of each other.

TABLE 2

| | Glucose | Fructose | Total sugar | Ethanol | Glycerol |
|---|---|---|---|---|---|
| Sample | g/l | g/l | g/l | g/l | g/l |
| F EMS 16 NaCl D7 | 0.03 | 1.25 | 1.28 | 102.9 | 8.23 |
| F EMS 16 NaCl D7 | 0.01 | 0.24 | 0.25 | 101.6 | 8.48 |
| F EMS 4 Pyra D11 | 0.06 | 1.49 | 1.55 | 105.0 | 6.65 |
| F EMS 4 Pyra D11 | 0.95 | 8.02 | 8.97 | 100.9 | 6.38 |
| Nitrite Pyra 12 B8 | 0.18 | 3.58 | 3.76 | 98.8 | 11.31 |
| Nitrite Pyra 12 B8 | 0.42 | 5.55 | 5.97 | 96.7 | 11.08 |
| Nitrite Pyra 12 H3 | 21.02 | 51.82 | 72.84 | 85.8 | 10.55 |
| Nitrite Pyra 12 H3 | 10.90 | 34.44 | 45.34 | 77.2 | 10.86 |
| Nitrite Pyra 25 A1 | 0.70 | 4.77 | 5.47 | 101.6 | 8.73 |
| Nitrite Pyra 25 A1 | 0.76 | 4.85 | 5.61 | 100.9 | 8.74 |
| UV Pyra 17 H8 | 0.02 | 0.72 | 0.74 | 103.6 | 8.52 |
| UV Pyra 17 H8 | 0.15 | 3.20 | 3.35 | 102.2 | 8.42 |

In conclusion, it was seen in the tests that after 35 days the mutant “Nitrite Pyra 12 B8” provides almost complete fermentation and in both fermentation trials produced more than 11 g/l glycerol. This mutant corresponds to the strain deposited with the Leibnitz-Institut DSMZ-Deutsche Sammlung von Mikroorganismen and Zellkulturen GmbH under accession number DSM 29822.

Printout (original in electronic format)
(This sheet does not count as a sheet in the International Application and is not part thereof)

| PCT | | |
|---|---|---|
| 0-1 | Form PCT/RO/134 | |
| | Indications Relating to Deposited Microor- | |
| | ganism or Other Biological Material | |
| 0-1-1 | Prepared with | PCT Online Filing |
| | | Version 3.5.000.244e MT/FOP |
| | | 20141031/0.20.5.20 |
| 0-2 | International application No. | PCT/EP2016/052437 |
| 0-3 | Applicant's or agent's file reference | 140557WO-ERS |
| 1 | The indications made below relate to the | |
| | microorganism and/or other biological | |
| | material referred to in the description on | |
| 1-1 | Page | 6 |
| 1-2 | Line | 19-24 |
| 1-3 | Identification of deposit | |
| 1-3-1 | Name of depositary institution | DSMZ Leibniz-Institut DSMZ-Deutsche |
| | | Sammlung von Mikroorganismen |
| | | und Zellkulturen GmbH (DSMZ) |
| 1-3-2 | Address of depositary institution | Inhoffenstr. 15,38124 Braunschweig, Germany |
| 1-3-3 | Date of deposit | Dec. 16, 2014 (16.12.2014) |
| 1-3-4 | Accession number | DSMZ 29822 |
| 1-4 | Additional indications | The “‘expert solution” is applied for in accordance |
| | | with Rule 13bis. 6 PCT for all countries/bureaux |
| | | for which this is applicable. |
| 1-5 | Designated states for which indications are | All designated states |
| | made | |

FOR RECEIVING OFFICE USE ONLY

| | | |
|---|---|---|
| 0-4 | This sheet was received with the Interna- | Yes |
| | tional Application | |
| | (iYes or No) | |
| 0-4-1 | Authorized Officer | Van Dooren, Luc |

FOR INTERNATIONAL BUREAU USE ONLY

| | |
|---|---|
| 0-5 | This sheet was received by the International |
| | Bureau on |
| 0-5-1 | Authorized officer |

1. A method for producing yeast mutants, comprising:
contacting at least one yeast strain a first mutagenesis step with a first mutagen and in a second mutagenesis step with a second mutagen, wherein the first and the second mutagen are different from each other and are selected from the group consisting of: nucleotide-alkylating agent, nucleotide-deaminating agent, and UV radiation; and
performing a first selection step between the first and second mutagenesis step and a second selection step after the second mutagenesis step, in which the mutants resulting from the preceding mutagenesis step are exposed to a selection factor selected from the groups consisting of:
(a) hypertonic medium and
(b) alcohol-dehydrogenase inhibitor.

2. The method according to claim 1, wherein the nucleotide-alkylating agent is selected from the group consisting of: dimethylsulphate (DMS), ethyl methanesulphonate (EMS), methyl methanesulphonate (MMS), 1methyl-3-nitro-1-nitrosoguanidine (MNNG), methylnitrosocyanamide (MNC), methylnitrosourea (MNU), and DNA methyltransferases.

3. The method according to claim 1, wherein the nucleotide-deaminating agent is selected from the group consisting of: anorganic nitrite salt, organic nitrite salt, and nitrous acid.

4. The method according to claim 1, wherein the first mutagen is a nucleotide-alkylating agent or a nucleotide-deaminating agent.

5. The method according to claim 1, wherein the first mutagen is a nucleotide-alkylating agent and the second mutagen is a nucleotide-deaminating agent or UV radiation.

6. The method according to claim 1, wherein the hypertonic medium is obtained by addition of a substance selected from the group consisting of: chlorides and sulphates of sodium, potassium, magnesium, calcium and sugar, including fructose and glucose and sugar alcohol, including sorbitol and mannitol.

7. The method according to claim 1, wherein the alcohol dehydrogenase inhibitor is selected from the group consisting of: pyrazole, 3-methylpyrazole, 4-methylpyrazole, and acetyl salicylic acid.

8. The method according to claim 1, wherein one of the selection factors is selected from hypertonic medium and one of the selection factors from alcohol-dehydrogenase inhibitor.

9. The method according to claim 1, further comprising performing a test step after the first selection step, in which intermediate yeast mutants obtained after the first selection step are tested for whether they produce more glycerol during an ethanolic fermentation than the initially used yeast strain under the same conditions, whereby only such intermediate yeast mutants to which this applies are subjected to the second mutagenesis step and the second selection step.

10. A yeast mutant obtained by a method according to claim 1 and deposited at the Leibniz-Institut DSMZ-Deutsche Sammlung von Mikroorganismen and Zellkulturen GmbH under accession number DSM 29822.

11. A yeast mutant obtained by a method according to claim 1, wherein during fermentation of must with 90° Oe (21.6% Brix) and a NOPA value=107 mg/l at a dosing of 4×106/ml, after 35 days at a fermentation temperature of 15-25° C. the mutants produce a wine with the following proportions of ethanol and glycerol:

| | | |
|---|---|---|
| Ethanol | 70-150 | g/l |
| Glycerol | 10-20 | g/l |

12. A method comprising producing an alcoholic beverage using the yeast mutant according to claim 10.

13. The method according to claim 12, wherein the alcoholic beverage is produced from grape must.
